(19) United States
(12) Patent Application Publication
(10) Pub. No.: US 2008/0293820 A1
(43) Pub. Date: Nov. 27, 2008

(54) METHODS FOR IMPROVING PHYSICAL FUNCTION IN FIBROMYALGIA

(75) Inventors: Srinivas Rao, Encinitas, CA (US); Michael Gendreau, Poway, CA (US); Jay Kranzler, La Jolla, CA (US)

Correspondence Address: DARBY & DARBY P.C., P.O. BOX 770, Church Street Station, New York, NY, 10008-0770, US

(73) Assignee: Cypress Bioscience, Inc., San Diego, CA (US)

(21) Appl. No.: 12/125,302

(22) Filed: May 22, 2008

Related U.S. Application Data
(60) Provisional application No. 60/939,548, filed on May 22, 2007.

(51) Int. Cl. A61K 31/165

(52) U.S. Cl. 514/620

(57) ABSTRACT

Methods for improving physical function in fibromyalgia syndrome by administering an NSRI such as milnacipran as disclosed.

This application claims the benefit of U.S. Provisional Application No. 60/939,548, filed May 22, 2007, the entire disclosure of which is hereby incorporated by reference.

### FIELD OF THE INVENTION

The present invention relates to a method for improving physical function in fibromyalgia by administering an NSRI, such as milnacipran, or a pharmaceutically acceptable salt thereof.

### BACKGROUND OF THE INVENTION

Fibromyalgia, also known as fibromyalgia syndrome (FMS) is a common systemic rheumatologic disorder estimated to affect 2% to 4% of the population, second in prevalence among rheumatologic conditions only to osteoarthritis. Wolfe et al., Arthritis Rheum. 1990; 33(2):160-172; Wolfe et al., Arthritis Rheum. 1995; 38(1):19-28. Fibromyalgia is associated with a reduced threshold for pain, generally identified by an increased sensitivity to pressure all over the body, and is often accompanied by fatigue, sleep disturbance, and morning stiffness. Other common symptoms include headache, migraine, variable bowel habits, diffuse abdominal pain, and urinary frequency. The diagnostic criteria for fibromyalgia require not only a history of widespread pain, but also the finding of tenderness on physical examination (“tender points”). In order to fulfill the criteria for fibromyalgia established in 1990 by the American College of Rheumatology (ACR), an individual must have both widespread pain involving all four quadrants of the body as well as the axial skeleton, and the presence of 11 of 18 tender points on examination. Wolfe et al., Arthritis Rheum. 1990; 33(2):160-172.

While there has been some suggestion that FMS may represent a form of somatization disorder, there is increasing evidence and acceptance that FMS is a medical problem, reflecting a generalized heightened perception of sensory stimuli. The abnormality is thought to occur within the central nervous system (CNS) rather than peripherally, and the proposed pathophysiological defect is termed “central sensitization”. Clauw D J and Chrousos G P, Neuroimmunomodulation 1997; 4(3):134-153; Yunas M B, J Rheumatol. 1992; 19(6):846-850; Bradley et al., Curr Rheumatol Rep. 2000; 2(2):141-148; Simms R W, Am J Med Sci. 1998; 315(6):346-350. FMS patients typically suffer from both allodynia (perceiving pain even from a non-painful stimulus such as light touch) and hyperalgesia (an augmentation of pain processing in which a painful stimulus is magnified and perceived with higher intensity than it would be by a normal volunteer). Mountz et al., Arthritis & Rheumatism 1995; 38(7):926-938; Arroyo J F and Cohen M L, J Rheunatol. 1993; 20(11):1925-1931. In this regard, there are many parallels in its clinical presentation and proposed underlying mechanisms with neuropathic pain, such as diabetic neuropathy and trigeminal neuralgia. Sindrup S H and T S Jensen, Pain 1999; 83(3):389-400; Woolf C J, Nature 1983; 306(5944):686-688; Woolf C J and R J Mannion, Lancet 1999; 353(9168):1959-1964. As a result, FMS is treated primarily within the medical model. It is most often diagnosed in the primary care setting, and almost half of the office visits are to internal medicine and family practice providers (1998 National Ambulatory Medical Care Survey). Visits to rheumatologists account for 16% of FMS patients' office visits. The remainder of visits are to a variety of tertiary care providers, including pain centers, physical medicine specialists, and psychiatrists.

Individuals with fibromyalgia suffer from a number of other symptoms, including a high incidence of recurrent non-cardiac chest pain, heartburn, palpitations, and irritable bowel syndrome. Wolfe, et al., Arthritis Rheum. 1990; 33(2):160-172; Mukerji et al., Angiology 1995; 46(5):425-430. Although the physiologic basis of these symptoms remains unclear, increasing evidence suggests that dysfunction of the autonomic nervous system is common in fibromyalgia and related illnesses. Clauw D J and Chrousos G P, Neuroimmunomodulation 1997; 4(3):134-153; Freeman R and Komaroff A L, Am J Med. 1997; 102(4):357-364. Prospective studies of randomly selected individuals with fibromyalgia have detected objective evidence of dysfunction of several visceral organs, including a 75% incidence of echocardiographic evidence of mitral valve prolapse, a 40-70% incidence of esophageal dysmotility, and diminished static inspiratory and expiratory pressures on pulmonary function testing. Lurie et al., Scand J Rehab Med. 1990; 22(3):151-155; Pellegrino et al., Arch Phys Med Rehab. 1989; 70(7):541-543. Neurally-mediated hypotension and syncope also appear to occur more frequently in individuals with fibromyalgia. Rowe et al., Lancet 1995; 345(8950):623-624.

Fibromyalgia is associated with high rates of disability, increased health care utilization, more frequent psychiatric consultations and a greater number of lifetime psychiatric diagnoses than controls.

A broad array of medications is used off-label in patients with FMS with varying degrees of success. Buskila D, Baillieres Best Pract Res Clin Rheumatol. 1999; 13(3):479-485; Leventhal L J, Ann Intern Med. 1999; 131(11):850-858; Lautenschlager J, Scand J Rheumatol Suppl. 2000:113:32-36. While antidepressants are the cornerstone of many treatment paradigms, other agents such as anti-convulsants, antispasticity agents, anxiolytics, sedatives, and opiates have been used. Non-steroidal anti-inflammatory drugs (NSAIDs) and acetaminophen are also used by a large number of patients (Wolfe et al., Arthritis Rheum. 1997; 40(9):1571-1579), even though peripheral inflammation has not been demonstrated (Clauw D J and Chrousos G P, Neuroimmunomodulation 1997; 4(3):134-153), and numerous studies have failed to confirm their effectiveness as analgesics in FMS. Goldenberg et al., Arthritis Rheum. 1986; 29(11):1371-1377; Yunus et al., J Rheumatol. 1989; 16(4):527-532; Wolfe et al., Arthritis Rheum. 2000; 43(2):378-385; Russell et al., Arthritis Rheum. 1991; 34(5):552-560; Quijada-Carrera et al., Pain 1996; 65(2-3):221-225. These agents do, however, provide an element of protection against other peripheral pain generators, such as osteoarthritis.

Antidepressants of all varieties represent a common form of therapy for many chronic pain states, including FMS. Sindrup S H and Jensen T S, Pain 1999; 83(3):389-400; Buskila D, Baillieres Best Pract Res Clin Rheumatol. 1999; 13(3):479-485; Leventhal L J, Ann Intern Med. 1999; 131(11):850-858; Lautenschlager J, Scand J Rheumatol Suppl. 2000; 113:32-36; Bennett R M, J Functional Syndromes 2001; 1(1):79-92. The majority of available antidepressants directly and/or indirectly increase the levels of 5-HT and/or NE in the CNS. Monoaminergic levels are increased either by inhibiting re-uptake (by blocking transport proteins) or interfering with the breakdown of the monoamine (by inhibiting the monoamine oxidase enzymes) after its release into the synaptic cleft.

Tricyclic Antidepressants (TCAs)

The TCAs most commonly employed in the treatment of FMS include amitriptyline, doxepin, and cyclobenzaprine. Buskila D, Baillieres Best Pract Res Clin Rheumatol. 1999; 13(3):479-485; Lautenschlager J, Scand J Rheumatol Suppl. 2000; 113:32-36; Bennett R M, J Functional Syndromes 2001; 1(1):79-92. While cyclobenzaprine is typically classified as a muscle relaxant rather than an antidepressant, it shares structural and pharmacological similarities with the TCAs, although its sedating qualities often override its usefulness in other applications. Kobayashi et al., Eur. J. Pharmacol. 1996; 311(1):29-35. TCAs block the re-uptake of both 5-HT and NE, but they favor NE re-uptake blockade, and the efficacy of TCAs can be interpreted to support the primacy of NE agonism for analgesic activity. However, TCA's additional anti-cholinergic, antihistaminergic, and α-adrenergic receptor blockade activities impart a wide assortment of undesirable side effects, which often compromise their tolerability and clinical acceptance. Kent J M, Lancet 2000; 355(9207):911-918.

TCAs have demonstrated moderate efficacy for the treatment of neuropathic pain conditions such as post-herpetic neuralgia and painful diabetic neuropathy. Max et al., Neurology 1988; 38(9):1427-1432; Max et al., N Eng J Med. 1992; 326(19):1250-1256; Watson et al., Neurology 1982; 32(6):671-673; Watson et al., Pain 1992; 48(1):29-36. Multiple studies of TCAs in the treatment of FMS support their use for this syndrome as well, and TCAs have frequently been used as the positive controls to which newer agents have been compared. Max et al., N Eng J Med. 1992; 326(19):1250-1256; Watson et al., Pain 1992; 48(1):29-36; Hannonen et al., Br J Rheumatol. 1998; 37(12):1279-1286; Goldenberg et al., Arthritis & Rheumatism 1996; 39(11):1852-1859.

Selective Serotonin Re-Uptake Inhibitors (SSRIs)

The SSRIs have revolutionized the treatment of depression with their improved side-effect profile secondary to more selective re-uptake inhibition. The SSRI agents fluoxetine, sertraline and citolopram have each been evaluated in randomized, placebo controlled trials in FMS. Goldenberg et al., Arthritis & Rheumatism 1996; 39(11):1852-1859; Wolfe et al., Scand J Rheum. 1994; 23(5):255-259; Anderberg et al., Eur J Pain 2000; 4(1):27-35; Norregaard et al., Pain 1995; 61(3):445-449. However, the results of these trials have been somewhat inconsistent, leaving much debate regarding the relative efficacy of the SSRIs, especially in comparison to TCAs.

Two placebo-controlled trials of citalopram in FMS patients were both convincingly negative. Anderberg et al., Eur J Pain, 2000; 4(1):27-35; Norregaard et al., Pain 1995; 61(3):445-449. This suggests that serotonergic enhancement alone is not sufficient to impart analgesia in the chronic pain setting. In fact, based on the evidence assembled to date, the SSRIs, as a class, are generally less efficacious than the TCAs in chronic pain states (Max et al., N Engl J Med. 1992; 326(19):1250-1256; Ansari A, Harv Rev Psych. 2000; 7(5):257-277; Atkinson et al., Pain 1999; 83(2):137-145; Jung et al., J Gen Intern Med. 1997; 12(6):384-389) although there are some exceptions (Saper et al., Headache 2001; 41(5):465-474).

Dual Re-Uptake Inhibitors

Dual re-uptake inhibitors (DRIs) are pharmacologically similar to TCAs (such as amitriptyline and doxepin), exhibiting dual activity upon serotonin and norepinephrine re-uptake. Sanchez C and Hytell J, Cell Mol. Neurobiol. 1999; 19(4):467-489. “Norepinephrine-serotonin reuptake inhibitor” (NSRI) and “serotonin-norepinephrine reuptake inhibitor” (SNRI) refer to subclasses of DRI's. DRI compounds that block the reuptake of norepinephrine preferentially are referred to as NSRI's, whereas those that preferentially block the reuptake of serotonin are referred to as SNRI's. These newer agents are generally devoid of significant activity at other receptor systems, resulting in diminished side effects and enhanced tolerability. Therefore, this class of compound may have significant potential for the treatment of FMS and/or other chronic pain conditions. SNRIs that are commercially available in the U.S. include venlafaxine and duloxetine. A number of such agents are in clinical development; these include milnacipran, bicifadine, viloxazine, LY-113821, SEP-227162, AD-337, and desvenlafaxine succinate (DVS-233).

One small, open-label trial of venlafaxine (EFFEXOR®) in 15 patients with FMS showed promising results. Dwight et al., Psychosomatics 1998; 39(1):14-17. Six of 11 completing patients had a positive response to venlafaxine, defined as 50% or greater improvement in two different measurements of overall pain. Insomnia was the most common side effect reported, requiring adjunctive medical therapy in 3 of 11 completing patients.

U.S. Pat. No. 6,602,911 describes the use of milnacipran for the treatment of FMS, the entire disclosure of which is incorporated herein by reference.

Opioids

Opiates exert their anti-nociceptive effects at various locations within both the ascending and descending pain pathways. Duale et al., Neuroreport 2001; 12(10):2091-2096; Besse et al., Brain Res. 1990; 521(1-2):15-22; Fields et al., Nature 1983; 306(5944):684-686; Yaksh et al., Proc Natl Acad Sci USA 1999; 96(14):7680-7686. Concerns regarding the use of opioids in chronic pain conditions have been raised. Bennett R M, J Functional Syndromes 2001; 1(1):79-92. Opioids are used by some in the clinical management of FMS, especially when other analgesics have failed to provide sufficient relief. Bennett R M, Mayo Clin Proc. 1999; 74(4):385-398.

To date, there have been no published reports of treatment of the physical component of fibromyalgia syndrome. Thus, there exists a need for an effective treatment of the physical component of fibromyalgia syndrome.

### SUMMARY OF THE INVENTION

The present invention relates to a method for improving physical function in fibromyalgia by administering an NSRI. In an exemplary embodiment, the present invention relates to a method for improving physical function in fibromyalgia by administering milnacipran, or a pharmaceutically acceptable salt thereof. In certain embodiments, the milnacipran, or a pharmaceutically acceptable salt thereof, is administered in an amount of 100 mg per day or 200 mg per day.

### BRIEF DESCRIPTION OF THE FIGURES

FIG. 1 summarizes the timeline of the study described in Example 1.

FIG. 2 is a flow chart showing the method of dosing patients in the study described in Example 2.

FIG. 3 summarizes the timeline of the study described in Example 2.

### DETAILED DESCRIPTION OF THE INVENTION

As used herein, the term “subject” or “patient” includes human and non-human mammals.

As used herein, “treatment” or “effective treatment” means to relieve, alleviate, delay, reduce, reverse, improve or prevent at least one symptom of fibromyalgia.

The term “dual reuptake inhibitor” (DRI) refers to a well-recognized class of antidepressant compounds that selectively inhibit reuptake of both norepinephrine and serotonin. “Norepinephrine-serotonin reuptake inhibitor” (NSRI) and “Serotonin-norepinephrine reuptake inhibitor” (SNRI) refer to subclasses of DRI's. DRI compounds that block the reuptake of norepinephrine preferentially are referred to as NSRI's, whereas those that preferentially block the reuptake of serotonin are referred to as SNRI's. Common DRI compounds include, but are not limited to, the SNRI's venlafaxine and duloxetine, and the NSRI's bicifadine and milnacipran.

NSRI compounds are described in detail in U.S. Pat. No. 6,602,911, the contents of which are hereby incorporated by reference in their entirety.

According to some embodiments, the present invention provides a method for improving physical function in fibromyalgia by administering an NSRI to a patient in need thereof.

In other exemplary embodiments, the NSRI is milnacipran, or a pharmaceutically acceptable salt thereof. In some embodiments, milnacipran may be administered as a hydrochloride salt: Z-2-aminomethyl-1-phenyl-N,N-diethylcyclopropanecarboxamide hydrochloride (chemical formula C₁₅H₂₃ClN₂O). In other embodiments, milnacipran, or a pharmaceutically acceptable salt thereof may be administered as a mixture of the dextro- and levrogyral enantiomers, e.g., as a mixture that includes more of one enantiomer or as a racemic mixture. In some embodiments, milnacipran, or a pharmaceutically acceptable salt thereof may be administered in an enantiomerically pure form (e.g., as the pure dextro- or pure levrogyral enantiomer). Unless otherwise indicated, milnacipran can include all stereoisomeric forms, mixtures of stereoisomeric forms, diastereomeric forms, and pharmaceutically acceptable salts thereof, including both enantiomerically pure forms of milnacipran as well as mixtures of milnacipran enantiomers. Methods for separating and isolating the dextro- and levrogyral enantiomers of milnacipran and other NE 5-HT SNRI compounds are well-known (see, e.g., Grard et al., 2000, Electrophoresis 2000 21:3028-3034).

Milnacipran

Milnacipran is an NSRI, i.e., a dual noradrenaline and serotonin re-uptake inhibitor that preferentially blocks reuptake of norepinephrine. Milnacipran is a CIS-(dl) racemate (Z form) composed of two (l- and d-) enantiomers. The chemical name of milnacipran's hydrochloride salt is: Z-2-aminomethyl-1-phenyl-N,N-diethylcyclopropanecarboxamide hydrochloride (C₁₅H₂₃ClN₂O).

Adverse events associated with milnacipran administration include: nausea, vomiting, headache, tremulousness, anxiety, panic attack, palpitations, urinary retention, orthostatic hypotension, diaphoresis, chest pain, rash, weight increase, back pain, constipation, diarrhea, vertigo, increased sweating, agitation, hot flushes, fatigue, somnolence, dyspepsia, dysuria, dry mouth, abdominal pain, and insomnia. Due to the high incidence of adverse events, patients often do not tolerate high-dose milnacipran. The present invention encompasses the discovery that the physical component of FMS can unexpectedly be treated by the administration of milnacipran.

Milnacipran monotherapy for the treatment of fibromyalgia and/or symptoms associated with fibromyalgia was previously described in a Phase II trial of 125 fibromyalgia patients. See, e.g., co-pending U.S. application Ser. No. 10/678,767, the contents of which are hereby incorporated by reference in their entirety. In this study, milnacipran was administered once or twice daily in a dosage escalation regimen to a maximum dose of 200 mg/day. Treatment with milnacipran provided a wide range of beneficial effects on the signs and symptoms of FMS. Twice-daily (BID) and once-daily (QD) dosing of milnacipran were approximately equally effective on fatigue, mood, global wellness, and function. Twice-daily dosing was better tolerated than QD dosing, and was more effective in treating pain than QD dosing. The patient global impression of change (PGIC) outcome measure showed that over 70% of completers in both milnacipran treatment groups reported an improvement in their overall status, while only 10% reported worsening. In contrast, 40% of the placebo patients who completed the trial rated themselves as worse at endpoint. The differences between placebo and milnacipran on the PGIC were statistically significant, both in terms of a comparison of mean endpoint scores, as well as on a binary improved/not-improved basis. This Phase II trial showed that treatment with 100 mg BID milnacipran was an effective acute (short-term) therapy for the symptom of pain in FMS, and milnacipran dosed either once or twice daily had measurable beneficial effects on a wide range of symptoms of FMS, including fatigue (measured on the FIQ), pain (multiple measures), quality of life (multiple measures), and, potentially, mood (Beck instrument). The data, however, did not suggest that administration of milnacipran could be used to improve physical function in fibromyalgia.

Effective Dosages:

Pharmaceutical compositions suitable for use in the present invention include an NSRI (e.g., milnacipran) and a pharmaceutically acceptable carrier or excipient. The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and, more particularly, in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin, the entire disclosure of which is hereby incorporated by reference.

In some embodiments of the present invention, the active agent (e.g., milnacipran) is administered in a dose of greater than 50 mg/day. In other embodiments, the active agent is administered in a dose of between about 100 mg/day and about 200 mg/day. In some embodiments, the active agent is administered in a dose of about 100 mg/day. In further embodiments, the active agent is administered in a dose of about 200 mg/day. In further embodiments, the active agent is administered in a dosage escalation comprising (i) administering about 12.5 mg per day for a first period of time, (ii) administering about 25 mg per day (e.g., about 12.5 mg twice a day) for a second period of time, (iii) administering about 50 mg per day (e.g., about 25 mg twice a day) for a third period of time, and (iv) administering about 100 mg per day (e.g., about 50 mg twice a day) for a fourth period of time. In another embodiment, the dosage escalation further comprises (v) administering about 200 mg per day (e.g., about 100 mg twice a day, about 50 mg four times a day) for a fifth period of time. In certain embodiments, the first period of time is 1 day, the second period of time is 2 days, the third period of time is four days. In other embodiments, each period of time is greater than 3 days. In additional embodiments, the active agent is administered for at least 3 months, e.g., for at least 6 months.

The route of administration of a pharmaceutical composition of the present invention can be, for example, oral, enteral, intravenous, and transmucosal (e.g., rectal). A preferred route of administration is oral.

Pharmaceutical compositions suitable for oral administration can be in the form of tablets, capsules, pills, lozenges, powders or granules, or solutions or dispersions in a liquid. Each of said forms will comprise a predetermined amount of a compound of the invention as an active ingredient. The composition in the form of a tablet can be prepared employing any pharmaceutical excipient known in the art for that purpose, and conventionally used for the preparation of solid pharmaceutical compositions. The examples of such excipients are starch, lactose, microcrystalline cellulose, magnesium stearate and binders, for example polyvinylpyrrolidone. Furthermore, an active compound can be formulated as controlled-release preparation, such as tablets comprising a hydrophilic or hydrophobic matrix.

A pharmaceutical composition of the present invention can be in the form of a capsule formulated using conventional procedures, for example by incorporation of a mixture of an active compound and excipients into a hard gelatin capsule. Alternatively, a semi-solid matrix of an active compound and high molecular weight polyethylene glycol can be formed and filled into hard gelatin capsules, or soft gelatin capsules can be filled with a solution of an active compound in polyethylene glycol or dispersion thereof in an edible oil. Powder forms for reconstitution before use (for example lyophilized powders) are also contemplated. Alternatively, oily vehicles for injection formulation can be used as well.

Liquid forms for parenteral administration can be formulated for administration by injection or continuous infusion.

Accepted routes of administration by injection are intravenous, intraperitoneal, intramuscular and subcutaneous. A typical composition for intravenous injection comprises a sterile isotonic aqueous solution or dispersion, including, for example, an active compound and dextrose or sodium chloride. Other examples of suitable excipients are lactated Ringer solution for injections, lactated Ringer solution for injections with dextrose, Normosol-M with dextrose, acylated Ringer solution for injections. The injection formulation can optionally include a co-solvent, for example polyethylene glycol, chelating agent, for example ethylenediaminotetraacetic acid; stabilizing agent, for example cyclodextrin; and antioxidant, for example sodium pyrosulfate.

The milnacipran dosage may be administered once per day or in divided doses that are given two or more times per day. In one embodiment, the milnacipran is administered twice a day. The amount of milnacipran administered to practice the methods of the present invention can vary depending on the subject being treated, the severity of the affliction, the manner of administration and the judgment of the prescribing physician.

Combination Therapy:

According to the present invention, milnacipran, or a pharmaceutically acceptable salt thereof, can be administered adjunctively with other active compounds for the long-term treatment of fatigue as the primary symptom of FMS. Other active compounds according to the invention include, for example, antidepressants, analgesics, muscle relaxants, anorectics, stimulants, antiepileptic drugs, beta blockers, and sedative/hypnotics. Specific examples of compounds that can be adjunctively administered include, but are not limited to, modafinil, gabapentin, pregabalin, pramipexole, 1-DOPA, amphetamine, tizanidine, clonidine, tramadol, morphine, tricyclic antidepressants, codeine, cambamazepine, sibutramine, valium, trazodone, caffeine, nicergoline, bifemelane, propranolol, and atenolol, and combinations thereof. In other embodiments of the present invention, milnacipran, is adjunctively administered pregabalin, gabapentin, pramipexole.

As used herein, adjunctive administration includes simultaneous administration of the compounds in the same dosage form, simultaneous administration in separate dosage forms, and separate administration of the compounds. For example, milnacipran can be simultaneously administered with valium, wherein both milnacipran and valium are formulated together in the same tablet. Alternatively, milnacipran can be simultaneously administered with valium, wherein both the milnacipran and valium are present in two separate tablets. In another alternative, milnacipran can be administered first followed by the administration of valium, or vice versa.

The following examples are merely illustrative of the present invention and should not be construed as limiting the scope of the invention in any way as many variations and equivalents that are encompassed by the present invention will become apparent to those skilled in the art upon reading the present disclosure.

### EXAMPLES

### Example 1

### A Multi-Center Double-Blind, Randomized, Placebo-Controlled Study of Milnacipran for the Treatment of Fibromyalgia

The primary objective of this study was to demonstrate safety and efficacy, both clinical and statistical, of milnacipran in the treatment of the fibromyalgia syndrome. The primary outcome was a composite responder analysis assessing response rate at weeks 14 and 15, and the secondary analysis assessed response rate at weeks 26 and 27.

Other objectives of this study were to:

1. compare statistical and clinical efficacy of 100 mg/day and 200 mg/day milnacipran in the treatment of the fibromyalgia syndrome based on each component of the composite responder analysis, as well as on a number of additional secondary endpoints including physical function, fatigue, sleep and mood, and cognition; and

2. establish and compare the safety profiles of 100 and 200 mg milnacipran daily in patients with FMS.

Methodology

This was a multi-center, randomized, double-blinded, placebo-controlled three-arm study, which enrolled 888 patients who met the 1990 ACR criteria for fibromyalgia syndrome as well as the more detailed admission criteria outlined in the protocol.

Patients recorded baseline symptoms for the first two weeks after washing off anti-depressants, benzodiazepines, and certain other drugs that could potentially interfere with efficacy measurements.

Patients were randomized to receive either placebo, 100 mg/day milnacipran, or 200 mg/day milnacipran in a ratio of a 1:1:2. All randomized medications (placebo and milnacipran) were administered in a split-dose (BID) fashion. The doses were administered in a dose escalation regimen as outlined below:

Step 1: 12.5 mg 1 day (12.5 mg pm)

Step 2: 25 mg 2 days (12.5 mg am, 12.5 mg pm)

Step 3: 50 mg 4 days (25 mg am, 25 mg pm)

Step 4: 100 mg 7 days (50 mg am, 50 mg pm)

Step 5: 200 mg 7 days (100 mg am, 100 mg pm).

All patients were scheduled to receive a total of 24 weeks of milnacipran or placebo after the 3 weeks of dose escalation steps, for a total of 27 weeks of milnacipran or placebo exposure.

Patients were required to complete electronic diary, as well as additional paper assessments as described in the schedule of study assessments.

Adverse event, physical examination, concomitant medication, vital sign and clinical laboratory data were collected as detailed in the schedule of study assessments.

Patients who successfully completed this double blind trial were eligible to participate in an open label trial for 15 to 28 additional weeks of therapy.

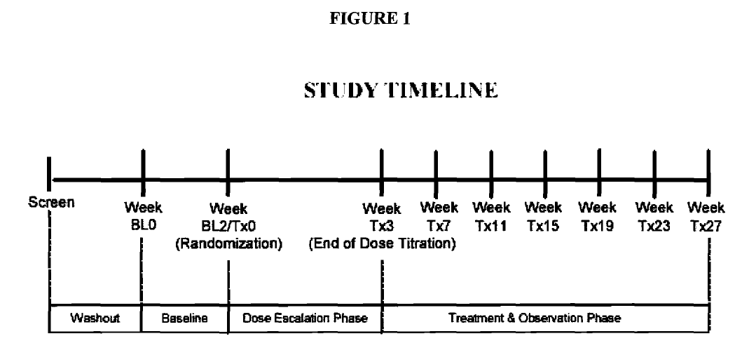

A timeline of the study is provided in FIG. 1.

### Assessments

Safety:

Safety of milnacipran was assessed by analyzing the frequency and severity of adverse events, changes in vital signs and clinical laboratory data collected during the study period.

Efficacy:

In addition to the daily completion of a proprietary electronic patient diary, the following assessments were obtained:

a. Primary Variables: patient global impression of change (PGIC) and the Short Form-36 (SF-36).

b. Psychological Screening at baseline: M.I.N.I.

c. Miscellaneous status assessments: periodically, as described in the schedule of evaluations: BDI, sleep quality scale, and the ASEX.

d. FMS Status Assessments: Patient pain 24 hour and 7 day recall VAS, the SF-36,

Multiple Ability Self-report Questionnaire (MASQ, cognitive function), the Multidimensional Health Assessment Questionnaire (MDHAQ) and the Multidimensional Fatigue Inventory (MFI). Diary assessments include current pain (morning, random daily, and evening reports); daily recall pain (morning report); medications taken (evening report); overall pain past week (weekly report), overall fatigue in the last week (weekly report), and the extent that pain kept the patient from caring for themselves (weekly report).

The SF-36 is a multi-purpose, short-form health survey. It yields an 8-scale profile of functional health and well-being scores, psychometrically-based physical and mental health summary measures, and a preference-based health utility index (Ware J E, Snow K K, Kosinski M, Gandek B. SF-36® Health Survey Manual and Interpretation Guide. Boston, Mass.: New England Medical Center, The Health Institute, 1993). The SF-36 provides a measure of a patient's functional impairment due to fatigue (i.e., how fatigue affects daily living activities of a patient). The SF-36 has proven useful in surveys of general and specific populations, comparing the relative burden of diseases, and in differentiating the health benefits produced by a wide range of different treatments.

The MASQ is a brief self-report questionnaire, which includes 5 cognitive domains: language ability, visuo-perceptual ability, verbal memory, visual memory, and attention/concentration (Seidenberg et al., J Clin & Exp Neuropsychology 1994; 16:93-104). The MASQ has been validated in both normal subjects and patient groups having cognitive difficulties in the assessment domains.

### Statistical Analysis

Efficacy:

The primary endpoint of this study was a composite responder analysis implementing analysis of three domains of interest, evaluated at 24 weeks as the primary analysis, and 12 weeks as the secondary analysis. The domains measured were:

1) pain (measured by an electronic diary as a daily recall pain score, calculated to weekly average scores)

2) patient global (measured by the PGIC, 1-7 scale)

3) physical function (measured by the SF-36 PCS).

For the primary analysis, the pain domain score was determined by a calculation that compared the average of treatment weeks 14 and 15 to the two baseline weeks, and treatment weeks 26 and 27 vs. baseline for the secondary analysis. The last observation was carried forward if neither the week 14 nor week 15 (or week 26/27) patient self-reported pain score is available to compare to the baseline value.

The binary response rate for placebo (based on the composite endpoint) in this study was expected to be in the range of 10-13%, with a milnacipran response rate in the active arm(s) expected in the 27-29% range on an ITT/LOCF basis. Based on these response rate assumptions, 125 patients randomized per arm (250 for high dose group) has been calculated to be the maximum sample size required (90% power). Secondary analyses included total area under the curve of pain intensity, and patient-reported weekly pain recall at the clinic visits as well as the FMS status assessments, and QOL measures.

### Results

A responder was defined as a subject who experienced a greater than 30% reduction in pain from baseline and improvement on the PGIC.

At three months, the percentage of responders was: 35.44% (56/158) in the placebo group; 53.33% (72/135) (p=0.001) in the milnacipran 100 mg/day group; and 55.00% (143/260) (p<0.001) in the milnacipran 200 mg/day group. At six months, the percentage of responders was: 32.86% (46/140) in the placebo group; 49.59% (60/121) (p=0.002) in the milnacipran 100 mg/day group; and 51.74% (119/230) (p<0.001) in the milnacipran 200 mg/day group. See Table 1 for a summary of the results in the Intent-to-Treat Population and Table 2 for a summary of the Last Observation Carried Forward (LOCF), Baseline Observation Carried Forward (BOCF) and study completer (OC) populations. LOCF is an analysis in which observations are carried forward to the last time point for patients who dropped out. The LOCF analysis treats the carried-forward data as observed data at the last time point. BOCF is an analysis that requires the patient remain active in the trial to be evaluated for response. If a patient withdraws from the trial for any reason they are classed as a non-responder irregardless of their pain and global scores at the time of withdrawal.

TABLE 1
Analysis of Responders for the Treatment of the Pain of Fibromyalgia
during Treatment Weeks 14-15 and 26-27 (Observed Cases)
Intent-to-Treat Population

| | | | | Milnacipran |
|---|---|---|---|---|
| | | Placebo | Milnacipran | 200 mg |
| | Statistic | (N = 223) | 100 mg (N = 224) | (N = 441) |
| Baseline | n | 223 | 224 | 441 |
| pain | mean | 68.37 | 68.32 | 69.41 |
| | SD | 11.98 | 11.54 | 11.85 |
| | SEM | 0.80 | 0.77 | 0.56 |
| | median | 66.5 | 67.9 | 69.1 |
| | min, max | 50, 100 | 41, 100 | 47, 99 |
| Treatment | n | 158 | 135 | 260 |
| weeks 14-15 | m (% = m/n) | 56 (35.44) | 72 (53.33) | 143 (55.00) |
| | odds ratio | | 2.10 | 2.20 |
| | 95% CI | | (1.31, 3.36) | (1.46, 3.31) |
| | p-value | | 0.002 | <0.001 |
| Treatment | n | 140 | 121 | 230 |
| weeks 26-27 | m (% = m/n) | 46 (32.86) | 60 (49.59) | 119 (51.74) |
| | odds ratio | | 1.96 | 2.20 |
| | 95% CI | | (1.18, 3.26) | (1.42, 3.41) |
| | p-value | | 0.009 | <0.001 |

TABLE 2
Summary of Composite Responder Rate

| | Pain Composite Resp | | | | | |
|---|---|---|---|---|---|---|
| | 3 month | | | 6 month | | |
| | placebo | 100 mg | 200 mg | placebo | 100 mg | 200 mg |
| | N = 223 | N = 224 | N = 441 | N = 223 | N = 224 | N = 441 |
| Primary | 27.8% | 33.5% | 34.9% | 25.1% | 30.8% | 32.2% |
| Analysis | | p* = 0.187 | p* = 0.058 | | p* = 0.197 | p* = 0.053 |
| (LOCF) | | | | | ap* = 0.393 | ap* = 0.105 |
| Sensitivity | 25.1% | 32.1% | 32.4% | 20.6% | 26.8% | 27.0% |
| Analysis I | | p* = 0.094 | p* = 0.048 | | p* = 0.167 | p* = 0.067 |
| (BOCF) | | | | | ap* = 0.334 | ap* = 0.133 |
| Sensitivity | 25.56% | 32.% | 32.7% | 21.5% | 27.2% | 28.6% |
| Analysis II | | p* = 0.113 | p* = 0.056 | | p* = 0.197 | p* = 0.048 |
| | | | | | ap* = 0.394 | ap* = 0.095 |
| Sensitivity | 25.1% | 32.1% | 32.4% | 22.9% | 29.5% | 29.9% |
| Analysis | | p* = 0.094 | p* = 0.048 | | p* = 0.120 | p* = 0.051 |
| III | | | | | ap* = 0.241 | ap* = 0.102 |
| OC | n* = 158 | n = 135 | n = 260 | n = 140 | n = 121 | n = 230 |
| Analysis | 35.4% | 53.3% | 55.0% | 32.9% | 49.6% | 51.7% |
| | | p* = 0.002 | p* < 0.001 | | p* = 0.009 | p* < 0.001 |

Summary of Individual Component Responder Rate

| | Pain Composite Resp, 3 month | | | Pain Composite Resp, 6 month | | |
|---|---|---|---|---|---|---|
| | pbo | 100 mg | 200 mg | pbo | 100 mg | 200 mg |
| Primary | 27.8% | 33.5% | 34.9% | 25.1% | 30.8% | 32.2% |
| Analysis | | p* = 0.187 | p* = 0.058 | | p* = 0.197 | p* = 0.053 |
| (LOCF) | | | | | ap* = 0.393 | ap* = 0.105 |
| Pain | 31.4% | 35.7% | 38.3% | 28.7% | 35.7% | 35.4% |
| (LOCF) | | p = 0.321 | p = 0.068 | | p = 0.110 | p = 0.072 |
| PGIC | 47.1% | 54.0% | 50.6% | 46.2% | 49.6% | 49.9% |
| (LOCF) | | p = 0.143 | p = 0.397 | | p = 0.476 | p = 0.368 |

*p-value: nominal p-value. ap = adjusted p-value at Step 2 for Hochberg's procedure (only valid if p-value is =<0.05 for 3-month pain for 200 mg compared to placebo at Step 1).
n = number of patients having adequate date for OC analysis (completers of landmark endpoint with observed values for responder assessment).

The mean baseline physical component scores (PCS) are shown in Table 3. The following domains were used to evaluate the PCS score: physical function (PFI), role physical (ROLP), bodily pain (PAIN), general health profile (GHP), invitality (VIT), social role (SOC), role emotional (ROLE) and mental health index (MHI). The PFI, ROLP, PAIN and GHP domains are positively weighted in the PCS calculation, and the VIT, SOC, ROLE and MHI domains are negatively weighted in the PCS calculation (Normative Value=50, SD=10).

TABLE 3
Baseline PCS Scores

| Placebo | 100 mg | 200 mg |
|---|---|---|
| 31.4 | 30.8 | 31.4 |

Table 4 shows the mean changes from baseline in PCS score at Tx 3, Tx 7, Tx 11 and Tx 15 (Observed Cohort).

TABLE 4
Mean Change from Baseline in PCS Score

| Treatment | | | | |
|---|---|---|---|---|
| Group | Tx 3 | Tx 7 | Tx 11 | Tx 15 |
| Placebo | 2.5 | 4.0 | 5.0 | 4.8 |
| 100 mg | 3.6 | 4.6 | 5.9 | 6.2 |
| 200 mg | 3.6 | 5.9 | 5.6 | 6.1 |

Table 5 shows the mean changes from baseline in the PFI, ROLP, PAIN and GHP SF-36 physical domain scores at Tx 3, Tx 7, Tx 11 and Tx 15 (Observed Cohort).

TABLE 5
Mean Change from Baseline in PFI, ROLP, PAIN and GHP SF-36 Physical
Domain Scores

| | PFI | | | | ROLP | | | | PAIN | | | | GHP | | | |
|---|---|---|---|---|---|---|---|---|---|---|---|---|---|---|---|---|
| | | | Tx | Tx | | | Tx | Tx | | | Tx | Tx | | | Tx | Tx |
| | Tx 3 | Tx 7 | 11 | 15 | Tx 3 | Tx 7 | 11 | 15 | Tx 3 | Tx 7 | 11 | 15 | Tx 3 | Tx 7 | 11 | 15 |
| Placebo | 4.8 | 6.5 | 8.4 | 7.2 | 10.1 | 15.6 | 16.4 | 16.5 | 6.9 | 11.9 | 12.7 | 12.4 | 3.9 | 6.6 | 6.3 | 6.1 |
| 100 mg | 6.4 | 7.6 | 10.6 | 11.4 | 12.3 | 16.4 | 18.6 | 19.3 | 11.8 | 15.0 | 19.4 | 18.1 | 5.9 | 6.3 | 8.1 | 7.8 |
| 200 mg | 8.2 | 11.0 | 10.7 | 11.9 | 13.0 | 19.0 | 18.4 | 19.9 | 12.5 | 18.6 | 16.8 | 19.3 | 6.0 | 8.5 | 7.7 | 8.2 |

The use of 100 mg or 200 mg milnacipran, or a pharmaceutically acceptable salt thereof, surprisingly and effectively improves physical function in patients with fibromyalgia when compared to patients treated with placebo.

### Example 2

### A Multicenter, Double-Blind, Randomized, Placebo-Controlled Monotherapy Study of Milnacipran for Treatment of Fibromyalgia

The primary objective of this study was to demonstrate the safety and efficacy, both clinical and statistical, of milnacipran in the treatment of fibromyalgia syndrome (FMS) or the pain associated with fibromyalgia. The primary outcome was a composite responder analysis assessing response rates of two doses (100 mg/day and 200 mg/day) of milnacipran as compared with placebo at Visit Tx15 (week 15).

Secondary objectives were (i) to compare statistical and clinical efficacy of 100 mg/day and 200 mg/day of milnacipran with placebo in the treatment of FMS, based on the time-weighted average of each component outcome of the composite responder endpoint from Visits Tx3 to Tx15 and (ii) to establish and compare the safety profiles of 100 mg/day and 200 mg/day milnacipran in patients with FMS.

### Methodology

This was a multicenter, randomized, double-blind, placebo-controlled three-arm study designed which enrolled 1196 patients who meet the 1990 ACR criteria for fibromyalgia syndrome (history of widespread pain and pain in 11 of 18 tender point sites on digital palpation), as well as the more detailed admission criteria outlined in the protocol.

Patients recorded baseline symptoms for the first two weeks after washing off anti-depressants, benzodiazepines, and certain other drugs that could potentially interfere with efficacy measurements.

Patients were randomized to receive placebo, 100 mg/day milnacipran or 200 mg/day milnacipran in a ratio of 1:1:1 (placebo=401 patients, 100 mg/day=399 patients, 200 mg/day=396 patients). The patients assigned to the two active treatment arms received a total of 12 weeks of stable-dose milnacipran exposure after the 3 weeks of dosage escalation steps, for a total of 15 weeks of drug exposure. All randomized medications (placebo and milnacipran) were administered twice a day (BID).

For the dose escalation period (Visits BL2/Tx0-Tx3), three blister cards were supplied, one for each week. On day one, in the evening, all three arms of the study received one large and one small capsule. In the case of the two active arms, the dose consisted of an active 12.5 mg capsule plus a placebo. In the case of the placebo arm, the dose consisted of one small and one large placebo capsule. On days two and three, the active arms each received one 12.5 mg active capsule plus a placebo capsule morning and evening and the placebo arm received two placebo capsules each morning and evening. For days 4-7, the active arms received one 25 mg active capsule plus a placebo capsule morning and evening and the placebo arm received 2 placebo capsules each morning and evening.

During the second week of the dose escalation period (i.e., days 8 through 14), patients in all three arms received only the larger 50 mg size capsules. Placebo patients received two large placebo capsules each time they took medication. Both the 100 mg and 200 active patients received one placebo and one active 50 mg capsule, morning and evening.

During the third week of the dose escalation phase, the placebo patients continued to receive two large placebo capsules, morning and evening. The 100 mg patients continued to receive one 50 mg active and one 50 mg placebo capsule, morning and evening. At this point, the 200 mg patients began receiving two 50 mg active capsules, morning and evening.

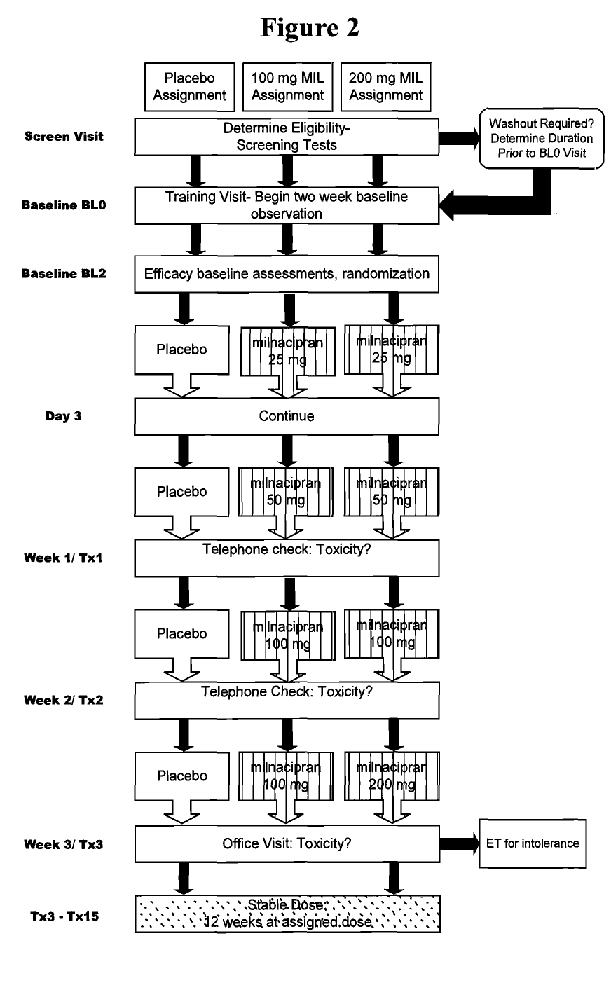

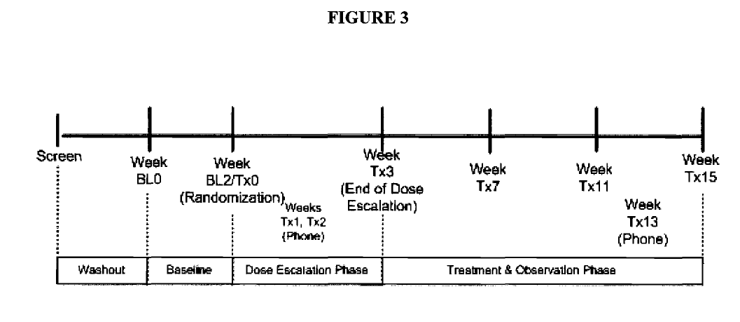

The dose escalation flow chart is shown in FIG. 2. A timeline of the study is provided in FIG. 3.

Patients were required to complete a proprietary electronic diary recording self-reported pain data as well as additional paper assessments as described in the schedule of study assessments.

Adverse event, physical examination, concomitant medication, vital sign, electrocardiogram (ECG) and clinical laboratory data were collected as detailed in the schedule of study assessments.

### Assessments

### Safety

Safety of milnacipran was assessed by analyzing the frequency and severity of adverse events (AEs), changes in vital signs, physical examination results, ECG, and clinical laboratory data collected during the study period.

### Efficacy

In addition to the daily completion of an electronic diary system, the following assessments were obtained:

(i) Primary Efficacy Assessments: Patient Global Impression of Change (PGIC) administered to patients at visits Tx3, Tx7, Txl 1 and Txl 5/ET; Physical Component summary of SF-36 (SF-36 PCS) administered to patients at visits BL2/TxO, Tx3, Tx7, Txl 1 and Tx15/ET;
(ii) Secondary Efficacy Assessments: Time weighted average (AUC) of weekly average PED morning recall pain score; PGIC and SF-36 PCS administered to patients at visits Tx3 to Txl5.
(iii) Additional Efficacy Measurements: The Fibromyalgia Impact Questionnaire (FIQ) total score and physical function, Beck Depression Inventory (BDI), the MOS-Sleep Index Scale, the Arizona Sexual Experiences Scale (ASEX), Patient pain 24 hour and 7 day recall VAS, the SF-36 individual domains, Patient Global Disease Status, Patient Global Therapeutic Benefit, the Multiple Ability Self-report Questionnaire (MASQ, cognitive function), the Multidimensional Health Assessment Questionnaire (MDHAQ), Multidimensional Fatigue Inventory (MFI), and diary assessments including current pain (morning, random daily, and evening reports); overall pain past week (weekly report), overall fatigue in the last week (weekly report), and the extent that pain kept the patient from caring for themselves (weekly report).

The primary efficacy assessment of this study was a composite responder status defined by three domains of interest evaluated at visit Tx15. The domains measured were

1) pain (measured by an electronic diary in the morning as a daily recall pain score);
2) patient global (measured by the PGIC, 1-7 scale);
3) physical function (measured by the SF-36 PCS).

The primary efficacy parameter for an indication in the treatment of pain of fibromyalgia was the composite responder status based on the morning recall pain as recorded in the PED and patient global as recorded on the PGIC at Visit Tx15.

The primary efficacy parameter for an indication in the treatment of FMS was the composite responder status based on two domains of pain and patient global as used above in the primary efficacy parameter for the treatment of the pain of fibromyalgia plus the additional domain of physical function as measured by the SF-36 PCS at Visit Txl5.

The secondary efficacy parameters were time-weighted average (AUC) of the weekly average PED morning recall pain scores for Weeks 4 through 15, PGIC, and SF-36 PCS for Visit Tx3 to Visit Tx15.

### Patient Physical Function

The physical function domain for response analysis was measured by the Physical Component Summary of SF-36 (SF-36 PCS). The SF-36 is a brief, well-established, self-administered patient questionnaire for the assessment of health status, functional status, and quality of life. The SF-36 measures eight domains of health status: physical functioning, role limitations due to physical problems, bodily pain, general health perceptions, energy/vitality, social functioning, role limitations due to emotional problems, and mental health. An SF-36 PCS score and a mental component summary (MCS) score can be calculated by combining and weighting the various individual scales. The PCS and MCS scores have been standardized to have a mean=50, SD=10 in the general healthy US population (see, e.g., Ware, J., M. Kosinski, and J. Dewey, How to Score Version 2 of the SF-36 Health Survey (Standard & Acute Forms). 3rd ed. 2000, Lincoln, R.I.: QualityMetric).

### Results

A patient was classified as a responder for the treatment of pain of fibromyalgia if he or she reached Visit Tx15 and satisfied the following criteria:
Greater than or equal to 30% in pain reduction from baseline;
PGIC rated as “much or very much improved,” (i.e., a score of 1 or 2 on the 1-7 scale at endpoint.)

A patient was classified as a responder for the treatment of FMS if he or she satisfied the responder criteria for the treatment of pain of fibromyalgia and the following additional criterion (at visit Tx15):
Improvement on the SF-36 PCS score from baseline by an amount at least equivalent to the minimal clinically important difference, as defined in the Statistical Analysis Plan.

Table 6 summarizes 3-month results for the Baseline Observation Carried Forward (BOCF), Last Observation Carried Forward (LOCF) and study completer (OC) populations. LOCF is an analysis in which observations are carried forward to the last time point for patients who dropped out. The LOCF analysis treats the carried-forward data as observed data at the last time point. BOCF is an analysis that requires that the patient remain active in the trial to be evaluated for response. If a patient withdrew from the trial for any reason, they were classified as a non-responder irregardless of their pain and global scores at the time of withdrawal.

TABLE 6
3-months

| | | | | | | | | OC, m/n = # of responders/# | | |
|---|---|---|---|---|---|---|---|---|---|---|
| | BOCF | | | | LOCF | | | of patients included | | |
| | | 100 mg | 200 mg | Global | | 100 mg | 200 mg | | 100 mg | 200 mg |
| | Placebo (401) | (399) | (396) | p-value | Placebo (401) | (399) | (396) | Placebo m/n | m/n | m/n |
| Composite | 66 | 91 | 98 | | 73 | 103 | 117 | 66/262 | 91/236 | 98/215 |
| Pain | (16.46%) | (22.81%) | (24.75%) | | (18.20%) | (25.81%) | (29.55%) | (25.19%) | (38.56%) | (45.58%) |
| | | 6.35% | 8.29% | | | 7.61% | 11.35% | | 13.37% | 20.39% |
| | | p = 0.025* | p = 0.004* | p = 0.004 | | p = 0.010 | p < 0.001 | | p = 0.001 | p < 0.001 |
| Pain | 101 | 124 | 119 | | 115 | 149 | 158 | 101/263 | 124/237 | 119/217 |
| Component | (25.19%) | (31.08%) | (30.05%) | | (28.68%) | (37.34%) | (39.90%) | (38.40%) | (52.32%) | (54.84%) |
| | | 5.89% | 4.86% | | | 8.66% | 11.22% | | 13.92% | 16.44% |
| | | p = 0.069 | p = 0.125 | | | p = 0.009 | p < 0.001 | | p = 0.002 | p < 0.001 |
| PGIC | 92 | 125 | 129 | | 100 | 138 | 151 | 92/289 | 125/263 | 129/255 |
| Component | (22.94%) | (31.33%) | (32.58%) | | (24.94%) | (34.59%) | (38.13%) | (31.83%) | (47.53%) | (50.59%) |
| | | 8.39% | 9.64% | | | 9.65% | 13.19% | | 15.70% | 18.76% |
| | | p = 0.008 | p = 0.002 | | | p = 0.003 | p < 0.001 | | <0.001 | <0.001 |
| Composite | 35 | 58 | 55 | | 39 | 65 | 65 | 35/262 | 58/236 | 55/215 |
| Syndrome | (8.73%) | (14.54%) | (13.89%) | | (9.73%) | (16.29%) | (16.41%) | (13.36%) | (24.58%) | (25.58%) |
| | | 5.81% | 5.16% | | | 6.56% | 6.68% | | 11.22% | 12.22% |
| | | p = 0.011* | p = 0.015* | p = 0.006 | | p = 0.006 | p = 0.003 | | p = 0.002 | p < 0.001 |
| SF36 PCS | 86 | 108 | 89 | | 102 | 129 | 109 | 86/290 | 108/263 | 89/255 |
| Component | (21.45%) | (27.07%) | (22.47%) | | (25.44%) | (32.33%) | (27.53%) | (29.66%) | (41.06%) | (34.90%) |
| | | 5.62% | 1.02% | | | 6.89% | 2.09% | | 11.40% | 5.24% |
| | | p = 0.063 | p = 0.586 | | | p = 0.029 | p = 0.348 | | p = 0.006 | p = 0.118 |

*Nominal p-value. All pair-wise comparisons to placebo for BOCF-based composite responder analyses are statistically significant (by the pre-specified multiple comparison procedure).

The mean baseline physical component scores (PCS) are shown in Table 7. The following domains were used to evaluate the PCS score: physical function (PFI), role physical (ROLP), bodily pain (PAIN), general health profile (GHP), invitality (VIT), social role (SOC), role emotional (ROLE) and mental health index (MHI). The PFI, ROLP, PAIN and GHP domains are positively weighted in the PCS calculation, and the VIT, SOC, ROLE and MHI domains are negatively weighted in the PCS calculation (Normative Value 50, SD=10).

TABLE 7
Baseline PCS Scores

| Placebo | 100 mg | 200 mg |
|---|---|---|
| 32.1 | 31.9 | 32.4 |

Table 8 shows the mean changes from baseline in PCS score at Tx 15 (Observed Cohort).

TABLE 8
Mean Change from Baseline in PCS Score at Tx 15

| Treatment | |
|---|---|
| Group | Tx 15 |
| Placebo | 2.9 |
| 100 mg | 5.0 |
| 200 mg | 3.9 |

Table 9 shows the mean changes from baseline in the PFI, ROLP, PAIN and GHP SF-36 physical domain scores at Tx 15 (Observed Cohort).

TABLE 9
Mean Change from Baseline in PFI, ROLP, PAIN and
GHP SF-36 Physical Domain Scores at Tx 15

| | PFI | ROLP | PAIN | GHP |
|---|---|---|---|---|
| | Tx 15 | Tx 15 | Tx 15 | Tx 15 |
| Placebo | 2.2 | 11.6 | 12.5 | 5.2 |
| 100 mg | 6.8 | 13.7 | 15.9 | 6.0 |
| 200 mg | 6.9 | 14.9 | 18.2 | 6.6 |

Table 10 shows the time-weighted average (AUC) of SF-36 Physical Component Summary (PCS) Scores for the 3-month treatment period (LOCF) (intent to treat population).

TABLE 10

| | | | Milnacipran | Milnacipran |
|---|---|---|---|---|
| | | Placebo | 100 mg | 200 mg |
| | | (N = 401) | (N = 399) | (N = 396) |
| Baseline | n | 401 | 397 | 393 |
| | Mean | 32.11 | 31.94 | 32.44 |
| | SD | 7.41 | 7.46 | 7.31 |
| | SEM | 0.37 | 0.37 | 0.37 |
| | Median | 31.9 | 31.8 | 32.8 |
| | Min, Max | 10, 56 | 13, 52 | 11, 53 |
| Visits | n | 401 | 397 | 393 |
| Tx3-Tx15 | Mean | 407.65 | 423.86 | 418.97 |
| | SD | 88.29 | 91.67 | 91.22 |
| | SEM | 4.41 | 4.59 | 4.58 |
| | Median | 405.2 | 418.2 | 419.2 |
| | Min, Max | 163, 672 | 185, 653 | 177, 658 |
| LS Mean (SE)* | | 412.02 | 425.22 | 420.38 |
| | | (4.545) | (4.540) | (4.697) |
| Difference | | | 16.78 | 8.36 |
| from Placebo* | | | | |
| 95% CI | | | (7.20, 26.36) | (−1.31, 18.03) |
| P-Value* | | | <0.001 | 0.090 |

SD = standard deviation,
SEM = standard error of the mean,
min = minimum,
max = maximum;
Analyses for comparison to placebo are based on an ANCOVA model with treatment group and study center as factors and baseline value as covariate.
LS Mean and SE for the placebo group are from the model comparing 200 mg group with placebo;
N = Number of patients who reached the endpoint with valid data during the respective time interval.

Table 11 shows the time-weighted average (AUC) of SF-36 Physical Component Summary (PCS) Scores for the 3-month treatment period (OC) (intent to treat population).

TABLE 11

| | | | Milnacipran | Milnacipran |
|---|---|---|---|---|
| | | Placebo | 100 mg | 200 mg |
| | | (N = 401) | (N = 399) | (N = 396) |
| Baseline | n | 286 | 262 | 251 |
| | Mean | 32.11 | 31.90 | 32.44 |
| | SD | 7.43 | 7.68 | 7.48 |
| | SEM | 0.44 | 0.47 | 0.47 |
| | Median | 31.9 | 31.5 | 32.6 |
| | Min, Max | 10, 56 | 15, 52 | 15, 53 |
| Visits | n | 286 | 262 | 254 |
| Tx3-Tx15 | Mean | 415.33 | 435.76 | 428.24 |
| | SD | 85.18 | 92.43 | 90.97 |
| | SEM | 5.04 | 5.71 | 5.71 |
| | Median | 415.2 | 432.8 | 428.4 |
| | Min, Max | 190, 672 | 209, 653 | 192, 658 |
| LS Mean (SE)* | | 418.29 | 437.91 | 429.46 |
| | | (5.152) | (5.207) | (5.374) |
| Difference | | | 20.22 | 11.17 |
| from Placebo* | | | | |
| 95% CI | | | (8.48, 31.95) | (−0.84, 23.18) |
| P-Value* | | | <0.001 | 0.068 |

SD = standard deviation,
SEM = standard error of the mean,
min = minimum,
max = maximum;
Analyses for comparison to placebo are based on an ANCOVA model with treatment group and study center as factors and baseline value as covariate.
LS Mean and SE for the placebo group are from the model comparing 200 mg group with placebo;
N = Number of patients who reached the endpoint with valid data during the respective time interval.

The use of 100 mg or 200 mg milnacipran, or a pharmaceutically acceptable salt thereof, surprisingly and effectively improves physical function in patients with fibromyalgia when compared to patients treated with placebo.

Each of the patent applications, patents, publications, and other published documents mentioned or referred to in this specification is herein incorporated by reference in its entirety, to the same extent as if each individual patent application, patent, publication, and other published document was specifically and individually indicated to be incorporated by reference.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A method of improving physical function in fibromyalgia said method comprising administering to a patient in need thereof a therapeutically effective amount of an NSRI.

2. The method of claim 1, wherein the NSRI is milnacipran, or a pharmaceutically acceptable salt thereof.

3. The method of claim 2, wherein about 100 mg/day of milnacipran, or a pharmaceutically acceptable salt thereof, is administered to the patient.

4. The method of claim 2, wherein about 200 mg/day of milnacipran, or a pharmaceutically acceptable salt thereof, is administered to the patient.

5. The method of claim 2, wherein the milnacipran, or a pharmaceutically acceptable salt thereof, is administered in divided doses.

6. The method of claim 3, wherein the milnacipran, or a pharmaceutically acceptable salt thereof, is administered as a 50 mg dose twice per day.

7. The method of claim 4, wherein the milnacipran, or a pharmaceutically acceptable salt thereof, is administered as a 50 mg dose four times per day.

8. The method of claim 1, further comprising adjunctively administering a second active compound for the treatment of cognitive dysfunction associated with FMS, wherein the second active compound is selected from the group consisting of an antidepressant, an analgesic, a muscle relaxant, an anorectic, a stimulant, an antiepileptic drug, a beta blocker, a sedative, a hypnotic, and combinations thereof.

9. The method of claim 8, wherein the second active compound is selected from the group consisting of modafinil, gabapentin, pregabalin, pramipexole, 1-DOPA, amphetamine, tizanidine, clonidine, tramadol, morphine, tricyclic antidepressants, codeine, cambamazepine, sibutramine, valium, trazodone, trazodone, caffeine, nicergoline, bifemelane, propranolol, atenolol, and combinations thereof.
